(19) United States
(12) Patent Application Publication
(10) Pub. No.: US 2017/0325800 A1
(43) Pub. Date: Nov. 16, 2017

(54) WOUND RETRACTOR AND SPECIMEN BAG ASSEMBLY

(71) Applicant: Covidien LP, Mansfield, MA (US)

(72) Inventor: Scott J. Prior, Shelton, CT (US)

(21) Appl. No.: 15/465,661

(22) Filed: Mar. 22, 2017

Related U.S. Application Data
(60) Provisional application No. 62/333,998, filed on May 10, 2016.

(51) Int. Cl. A61B 17/02; A61B 17/3211; A61B 17/34; A61B 17/32; A61B 17/00

(52) U.S. Cl. CPC A61B 17/0293; A61B 17/3423; A61B 17/3211; A61B 2017/00287; A61B 2017/320024; A61B 2017/3486; A61B 2017/0225

(57) ABSTRACT

A surgical apparatus of the present disclosure includes a wound retractor having a specimen bag attached thereto. The wound retractor includes a proximal ring, a distal ring, and a film extending therebetween. The specimen bag is attached to the distal ring of the wound retractor. Kits of the present disclosure include the surgical apparatus and a wound guard. Where the tissue specimen to be removed from the patient is too large for passing through an incision or wound, the wound guard is introduced into a lumen of the wound retractor. Mechanical devices such as scalpels or morcellators may then be introduced into the lumen of the wound guard and manipulated adjacent the wound guard to break up the tissue specimen without damaging the specimen bag or wound retractor. Methods for using the surgical apparatus and kits of the present disclosure are also provided.

### CROSS-REFERENCE TO RELATED APPLICATIONS

This application claims the benefit of and priority to U.S. Provisional Patent Application No. 62/333,998 filed May 10, 2016, the entire disclosure of which is incorporated by reference herein.

### TECHNICAL FIELD

The present disclosure relates generally to surgical apparatuses for use in minimally invasive surgical procedures, such as endoscopic and/or laparoscopic procedures, and more particularly, the present disclosure relates to a surgical apparatus including a wound protection device and a specimen retrieval bag assembly for accessing and collecting body tissue(s) and/or body fluid(s) during these procedures.

### BACKGROUND

Minimally invasive surgery, such as endoscopic surgery, reduces the invasiveness of surgical procedures. Endoscopic surgery involves surgery through body walls, for example, viewing and/or operating on the ovaries, uterus, gall bladder, bowels, kidneys, appendix, etc. There are many common endoscopic surgical procedures, including arthroscopy, laparoscopy, gastroentroscopy and laryngobronchoscopy, just to name a few. In these procedures, trocars are utilized for creating incisions through which the endoscopic surgery is performed. Trocar tubes or cannula devices are extended into and left in place in the abdominal wall to provide access for endoscopic surgical tools. A camera or endoscope is inserted through a trocar tube to permit the visual inspection and magnification of the body cavity. The surgeon can then perform diagnostic and/or therapeutic procedures at the surgical site with the aid of specialized instrumentation, such as forceps, graspers, cutters, applicators, and the like, which are designed to fit through additional cannulas. To protect the opening from accidental penetration by the surgical instruments, wound retractors and wound protectors are often placed across the opening.

When removing certain tissues from the body cavity, for example tumor tissue, it is important that the tumor tissue does not come into contact with healthy or uninvolved tissue, so as in this way to avoid metastasis and avoid harming the patient. Minimally invasive surgical procedures, however, may be limited where large size tumors or large masses of tissue have to be removed from a body cavity. If tumor tissue or tissue parts have to be removed, they may be introduced into an “extraction bag,” also referred to herein as a “specimen bag,” at the site where the tumor or diseased tissue has been detached from the surrounding tissue, after which the specimen bag is withdrawn from the body, normally through a trocar or similar device, thereby minimizing contact of the diseased tissue with healthy tissue.

In some instances, depending upon the volume of tissue being removed from the body, the tissue within the specimen bag must be broken up prior to removal from the body to allow for the specimen bag and its contents to pass through the opening used to conduct the endoscopic procedure.

Improved wound retractors and specimen bags for use in minimally invasive surgical procedures remain desirable.

### SUMMARY

The present disclosure is directed to surgical apparatuses and kits for use in minimally invasive surgery. The surgical apparatus includes a wound retractor having a specimen bag attached thereto, for placement of diseased tissue therein. In embodiments, the kit of the present disclosure includes the surgical apparatus and a wound guard for assisting in breaking up tissue prior to its removal from the body.

A kit of the present disclosure includes a wound retractor and specimen bag assembly, in combination with a wound guard. The wound retractor and specimen bag assembly includes a proximal ring, a distal ring, a film disposed between the proximal ring and the distal ring, and a specimen bag attached to the distal ring of the wound retractor.

In embodiments, the proximal ring of the wound retractor defines a generally circular opening and is deformable. Similarly, the distal ring of the wound retractor may also define a generally circular opening and is deformable.

In some embodiments, the film of the wound retractor defines an adjustable length between the proximal ring and the distal ring.

In certain embodiments, the specimen bag is attached to the distal ring of the wound retractor by a method such as adhesive bonding, welding, heat-sealing, and combinations thereof.

In some embodiments, the proximal ring, the distal ring, or both, possess a tether thereon.

In embodiments, the wound guard includes a distal portion defining a generally circular opening, a proximal portion defining a generally circular opening, and an elongate shaft defining a lumen between the distal portion and the proximal portion.

In certain embodiments, the wound guard possesses an anchoring balloon which is inflated to anchor the wound guard in a body opening.

In embodiments, the kit of the present disclosure also includes a grasping element for grasping tissue for removal from the specimen bag.

In some embodiments, the kit of the present disclosure also includes a device for breaking up tissue as it is removed from the specimen bag. Suitable devices for breaking up tissue as it is removed from the specimen bag include a scalpel, a morcellator, a knife, and/or combinations thereof.

Methods of the present disclosure include, in embodiments, introducing into a body opening a wound retractor and specimen bag assembly having a proximal ring, a distal ring, a film disposed between the proximal ring and the distal ring, and a specimen bag attached to the distal ring, by inserting the wound retractor and specimen bag assembly into a body cavity through the body opening. A tissue specimen is then passed through an opening defined by the proximal ring, the film and the distal ring of the wound retractor into the specimen bag, after which the proximal ring is removed from the body cavity through the body opening so that the film is adjacent tissue encompassing the body opening. The proximal ring is then pulled away from the distal ring such that the distal ring is brought into abutment with an inner surface of the tissue surrounding the body opening, and the proximal ring is rolled about itself such that the film is furled about the proximal ring to bring the proximal ring into abutment with an outer surface of the tissue. A wound guard is then introduced through the proximal ring into the body opening and the tissue specimen is removed from the specimen bag.

In embodiments, methods of the present disclosure further include rolling the proximal ring to retract the incision and bring the proximal ring into abutment with an outer surface of the tissue.

In some embodiments, methods of the present disclosure further include inflating an anchoring balloon on the wound guard to anchor the wound guard in the body opening.

In certain embodiments, methods of the present disclosure further include introducing a grasping element through the wound guard into the specimen bag and grasping the tissue specimen therein, and proximally pulling the grasping element while breaking up the tissue specimen as the tissue specimen passes through the wound guard upon its removal from the specimen bag.

In other embodiments, methods of the present disclosure further include breaking up the tissue specimen prior to removing the tissue specimen from the specimen bag. Breaking up the tissue specimen as it passes through the wound guard upon its removal from the specimen bag may include mechanically breaking up the tissue.

### BRIEF DESCRIPTION OF THE DRAWINGS

Various embodiments of the presently disclosed wound retractor and specimen bag assembly are described herein with reference to the drawings wherein:

FIG. 1 is a perspective view of components of a kit including an exemplary embodiment of the presently disclosed wound retractor and specimen bag assembly;

FIG. 2 is a perspective view of the specimen bag and wound retractor assembly shown in FIG. 1;

FIG. 3 is a cross-sectional view of a patient's body, showing a wound, incision, and tissue specimen to be removed from the body cavity;

FIG. 4A is a perspective, partial cross-sectional view showing the partial removal of the wound retractor and specimen bag assembly in FIG. 2 from a body cavity through an incision;

FIG. 4B is a perspective view of the wound retractor and specimen bag assembly shown in FIG. 2 in a body cavity, adjacent tissue to be removed from the body cavity;

FIG. 5 is a perspective view of the wound retractor and specimen bag assembly shown in FIG. 2 inserted into an incision in tissue and the specimen bag positioned within the body cavity;

FIG. 6 is a perspective, partial cross-sectional view of the wound retractor and specimen bag assembly shown in FIG. 2 with a wound guard and surgical instrumentation inserted through the wound retractor into the specimen bag; and

FIG. 7 is a perspective view the wound retractor and specimen bag assembly shown in FIG. 2 with the wound guard and surgical instrumentation inserted into the specimen bag as tissue is removed from the specimen bag.

### DETAILED DESCRIPTION

The present disclosure provides a wound retractor and specimen bag assembly for use in minimally invasive surgical procedures. As used herein with reference to the present disclosure, minimally invasive surgical procedures encompass laparoscopic procedures and endoscopic procedures, and refer to procedures utilizing scopes or similar devices having relatively narrow operating portions or a small incision in the skin.

The aspects of the present disclosure may be modified for use with various methods for retrieving tissue specimens during minimally invasive surgical procedures, sometimes referred to herein as minimally invasive procedures. Examples of minimally invasive procedures include, for example, cholecystectomies, appendectomies, nephrectomies, colectomies, splenectomies, and the like.

As used herein, the term distal refers to the portion of a surgical apparatus, including a wound retractor and specimen bag of the present disclosure, which is farthest from the user, while the term proximal refers to that portion of the surgical apparatus of the present disclosure which is closest to the user.

The wound retractor and specimen bag assembly of the present disclosure includes a wound retractor with a specimen bag affixed thereto. The wound retractor component includes a sleeve member possessing at least two rings, including a proximal ring and a distal ring, with a film extending between the two rings. The specimen bag component is attached to the distal ring of the wound retractor. In use, the entire surgical apparatus is passed through an incision and placed within a body cavity, and tissue to be removed therefrom, referred to in embodiments as a “tissue specimen”, is passed through the wound retractor component and placed within the specimen bag. The proximal ring of the wound retractor component is then removed from the body cavity and placed adjacent the skin on the outside of the body adjacent the incision, with the distal ring of the sleeve member and specimen bag remaining within the body, so that the film extends through the incision and is adjacent the tissue encompassing the incision into the body. The presently disclosed wound retractor and specimen bag assembly is suitable for use in any procedure where access to the interior of the body is limited to one or more relatively small incisions, as in minimally invasive procedures.

Kits of the present disclosure include both the wound retractor and specimen bag assembly described above, as well as a substantially rigid wound guard. The wound guard protects the assembly and a patient during morcellation of tissue with a scalpel or some other morcellation device as the tissue is removed from the specimen bag.

Embodiments of the present disclosure will now be described in detail with reference to the drawings, in which like reference numerals designate identical or corresponding elements in each of the several views. In the following description, well-known functions or constructions are not described in detail to avoid obscuring the present disclosure in unnecessary detail.

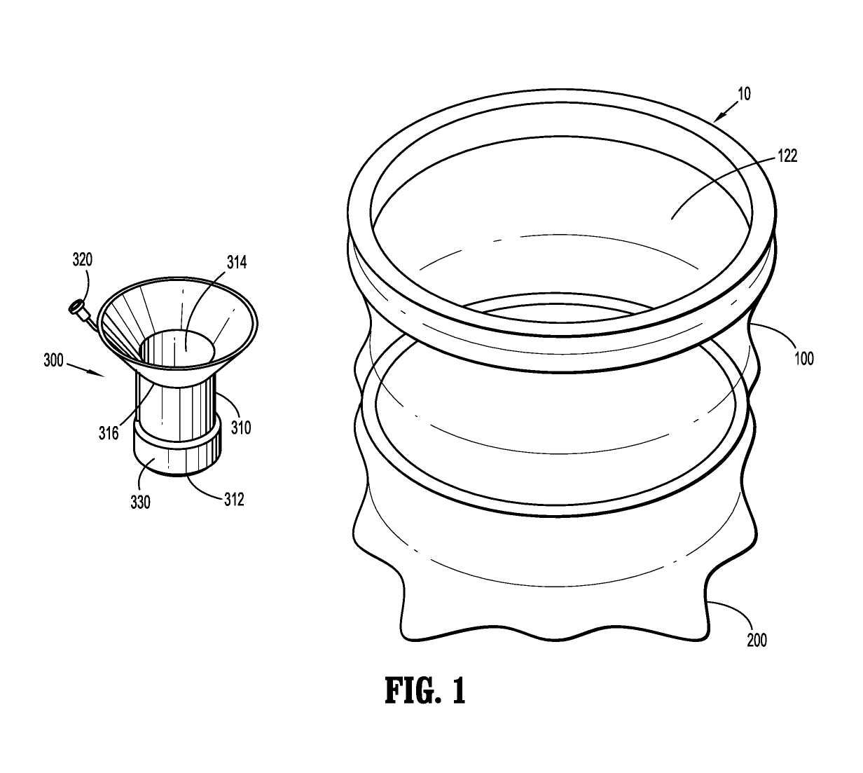

FIG. 1 illustrates a kit of the present disclosure including a wound retractor and specimen bag assembly 10 in accordance with the present disclosure. The wound retractor and specimen bag assembly 10 encompasses both a wound retractor 100 and a specimen bag 200. The kit of the present disclosure also includes a wound guard 300.

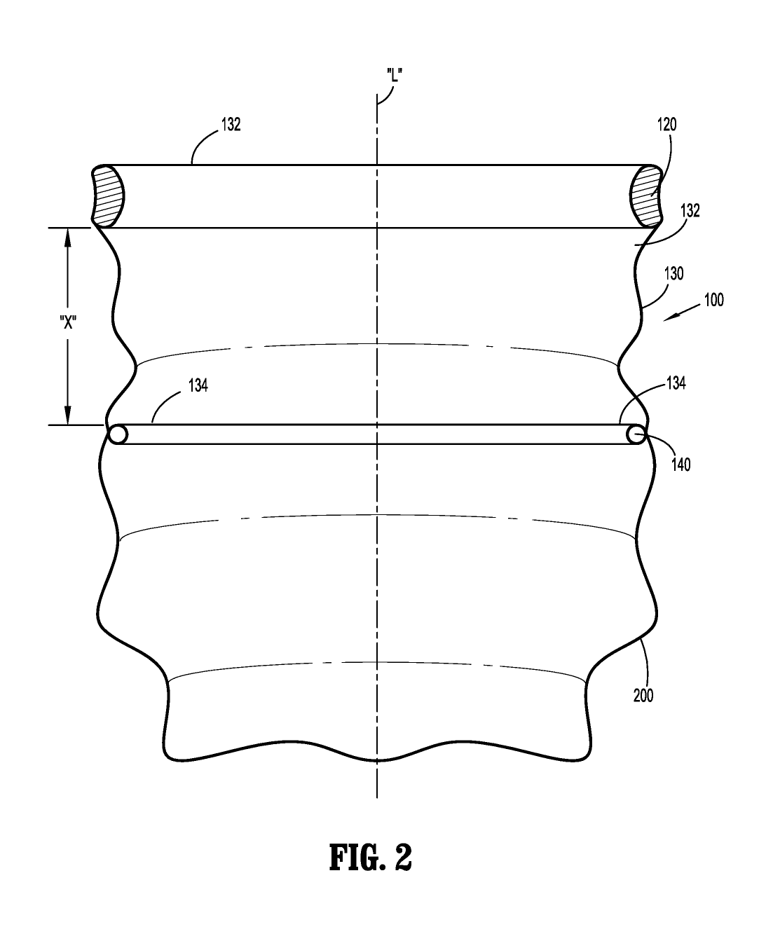

The wound retractor and specimen bag assembly 10, including the wound retractor 100 and the specimen bag 200, is adapted for insertion into wound “W” through an incision “I” (as illustrated in FIG. 2). The incision “I” may be a single incision, e.g., through the abdominal or peritoneal lining, or a naturally occurring orifice (i.e. mouth, anus, or vagina).

Referring also to FIG. 2, the wound retractor 100 includes a proximal ring 120, a distal ring 140, and a generally cylindrical film 130 disposed therebetween. The wound retractor and specimen bag assembly 10 further defines a longitudinal axis “L” shared by the proximal ring 120, the distal ring 140, and the film 130, as well as the specimen bag 200. It is envisioned that the proximal ring 120 and the distal ring 140 may each be detachably coupled or permanently attached to a proximal end portion 132 and a distal end portion 134, respectively, of the film 130 by any means within the purview of those skilled in the art, e.g., glue, suture, impulse welding, chemical or mechanical bonding, an over molding process, etc. In some embodiments, the proximal ring 120, film 130, and distal ring 140 are monolithically integrated such that the wound retractor 100 is a unitary structure. Attachment of the film 130 to the rings 120, 140 can be continuous around the rings 120, 140 or may be discontinuous, provided the rings 120, 140 are sufficiently secured to the film 130 to allow the film 130 to be rolled about the proximal ring 120 as described below.

The proximal ring 120 and the distal ring 140 are axially aligned along longitudinal axis “L” with the film 130 disposed therebetween, as noted above. The proximal ring 120 has a generally circular configuration that defines a circular opening 122 (see FIG. 1) and is rollable, such that the proximal ring 120 can be rolled towards or away from the distal ring 140 along the longitudinal axis “L”, which results in the film 130 being furled or unfurled about the proximal ring 120. The distal ring 140 also has a generally circular configuration that defines a generally circular opening (not shown).

It is envisioned that the proximal ring 120 and the distal ring 140 may be fabricated from resilient materials such that the proximal ring 120 and the distal ring 140 may temporarily deform into a generally oblong configuration during insertion of the wound retractor 100 through an incision “I” while reverting to a generally circular configuration during use. For example, thermoplastic polyurethanes sold under the name PELLETHANE®, offers flexibility and a wide range of hardnesses. The proximal ring 120, for example, may be fabricated from PELLETHANE® 2363-80A, PELLETHANE® 2363-90A, a 50/50 composition of PELLETHANE® 2363-80A and PELLETHANE® 2363-90A, or any alternatives known in the art. The distal ring 140 may be fabricated from, for example, PELLETHANE® 2363-90A for the extra small and small size, PELLETHANE® 2363-55D for the medium and large size, a 50/50 composition of PELLETHANE® 2363-90A and PELLETHANE® 2363-55D for the large size, or any alternatives within the purview of those skilled in the art. The proximal ring 120 and the distal ring 140 may be fabricated from the same or different materials.

The film 130 defines a generally cylindrical shape to form a lumen between the proximal ring 120 and the distal ring 140. The circular configuration of the proximal and distal rings 120 and 140, respectively, maintains the film 130 in an expanded state to maintain the lumen in a non-collapsed state. The film 130 is coupled to the proximal ring 120 at the proximal end portion 132 and is coupled to the distal ring 140 at the distal end portion 134, such that the film 130 does not slide or move relative to a surface of either the proximal ring 120 or the distal ring 140. The film 130 may be fabricated from a clear, non-elastomeric material, e.g., a polyurethane. Alternately, it is envisioned that the film 130 may be formed from a variety of materials including opaque and clear materials.

The length “X” between the proximal ring 120 and the distal ring 140 is adjustable. More specifically, the length “X” can be decreased by rolling the proximal ring 120 towards the distal ring 140 to furl the film 130 about the proximal ring 120. Similarly, the length “X” can be increased by rolling the proximal ring 120 away from the distal ring 140 to unfurl the film 130 from about the proximal ring 120. As discussed above, as the proximal ring 120 is rolled towards or away from the distal ring 140, the film 130 furls or unfurls about the proximal ring 120. It should be appreciated that as the film 130 is furled about the proximal ring 120, it reduces the length “X” of the film 130, so the tension in the film between the proximal ring 120 and distal ring 140 is increased to provide a radially outward force within the incision “I”.

It is envisioned that the wound retractor 100 may come in a variety of sizes to appropriately fit and accommodate a range of incision diameters. For example, for an extra small wound retractor, the outer diameter of the proximal ring 120 and the distal ring 140 may be about 2.5 inches; for a small wound retractor, the outer diameter of the proximal ring 120 and the distal ring 140 may be about 3.9 inches; for a medium sized wound retractor, the outer diameter of the proximal ring 120 and the distal ring 140 may be about 5.1 inches; for a large wound retractor, the outer diameter of the proximal ring 120 and the distal ring 140 may be about 7.5 inches; and for an extra-large wound retractor, the outer diameter of the proximal ring 120 and the distal ring 140 may be about 9.4 inches.

Specimen bags of the present disclosure are made of flexible and durable materials within the purview of those skilled in the art, in embodiments, polymeric materials. The specimen bags are inflatable and capable of allowing a surgeon to introduce cutting devices into the specimen bag to reduce the size of the tissue specimen therein, thereby facilitating removal of the specimen bag from the body. Materials used to form the specimen bags are antistatic, pyrogen-free, non-toxic and sterilizable. In embodiments, materials used to form the film portion of the wound retractor described above may be used to form the specimen bag. In other embodiments, the specimen bag is formed of materials that are different from those used to form the film of the wound retractor. The specimen bag may be opaque or clear.

As depicted in FIG. 2, the specimen bag 200 may be joined to the distal ring 140 of the wound retractor at the distal portion 134 of the film 130 by methods within the purview of those skilled in the art, including, but not limited to, adhesive bonding, welding, heat-sealing, combinations thereof, and the like. Alternatively, the specimen bag 200 can be integrally formed with the film 130, in embodiments of the same material.

Both the proximal ring 120 and the distal ring 140 may be collapsed from the generally circular configuration to a generally oblong configuration (not shown) for insertion, along with the specimen bag 200, through incision “I”. More specifically, as the practitioner squeezes opposing sides of the proximal ring 120 and the distal ring 140 radially inwards, the generally circular openings of the proximal ring 120 and the distal ring 140 are deformed from the generally circular configuration to a generally oblong configuration (not shown) such that the proximal ring 120 and the distal ring 140 assume a smaller profile for ease of insertion through incision “I”. Once inserted, the practitioner releases the proximal ring 120 and distal ring 140 and the resiliency of the material urges the proximal ring 120 and the distal ring 140 towards their generally circular configurations.

In embodiments, where the tissue specimen “T” within the specimen bag 200 is too large to pass through the wound retractor 100, the tissue may have to be broken up by a scalpel, a morcellator, or a similar device to facilitate removal of the tissue specimen “T” from the specimen bag 200. As used herein, the term morcellator refers to a surgical instrument for cutting, mincing up, liquefying, or morcellating, tissue into smaller pieces. Care should be taken not to damage the specimen bag to prevent resected tissue from entering into a body cavity, e.g., the abdominal cavity. Accordingly, in embodiments a wound guard 300 is provided to protect the wound retractor and specimen bag assembly and the patient during morcellation of the tissue specimen “T”.

The kit (FIG. 1) of the present disclosure includes a wound guard 300. The wound guard 300 has an elongate body 310 defining a lumen 314. The elongate body 310 has a distal portion 312 defining a generally circular opening and a proximal portion 316 defining a generally circular opening.

The wound guard 300 may be formed of hard, rigid materials. Suitable materials for forming the wound guard 300 include, for example, polyolefins such as polyethylene (including ultra high molecular weight polyethylene) and polypropylene including atactic, isotactic, syndiotactic, and blends thereof; polyethylene glycols (PEGs); polyethylene oxides; copolymers of polyethylene and polypropylene; polyisobutylene and ethylene-alpha olefin copolymers; fluorinated polyolefins such as fluoroethylenes, fluoropropylenes, fluoroPEGs, and polytetrafluoroethylene; polyamides; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers and copolymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; copolymers of vinyl monomers with each other and olefins such as ethylene-methyl methacrylate copolymers; acrylonitrile-styrene copolymers; acrylonitrile butadiene styrene resins; ethylene-vinyl acetate copolymers; alkyd resins; polycarbonates; polyoxymethylenes; polyphosphazines; polyimides; epoxy resins; aramids; silicones; and copolymers and combinations thereof.

The wound guard 300 also possesses an inflation port 320 for introducing inflation gases into an anchoring balloon 330 located at the distal portion 312 of the elongate shaft 310. The anchoring balloon 330 can be inflated to secure the distal portion 312 of the wound guard 300 within the body cavity.

The anchoring balloon 330 at the distal portion 312 of the elongate shaft 310 of the wound guard 300 may be inflated with any medical grade fluid, such as saline, CO₂, or any other suitable fluid using a syringe, mechanically or manually operated pump, or other means. The inflation port 320 for inflating the anchoring balloon 330 may be used with one-way valves, check valves, or any other valve arrangement for inflating the anchoring balloon 330.

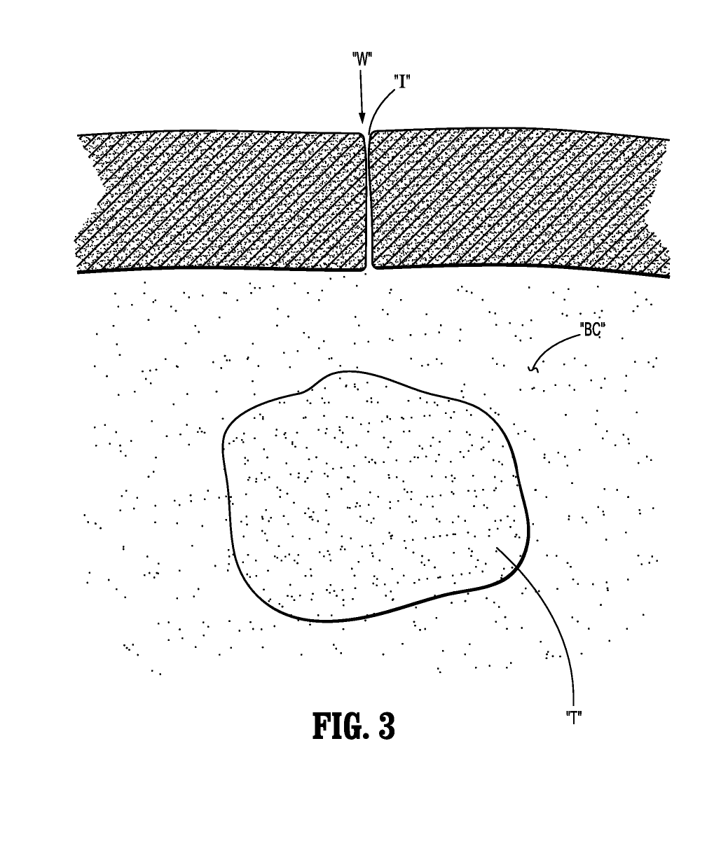

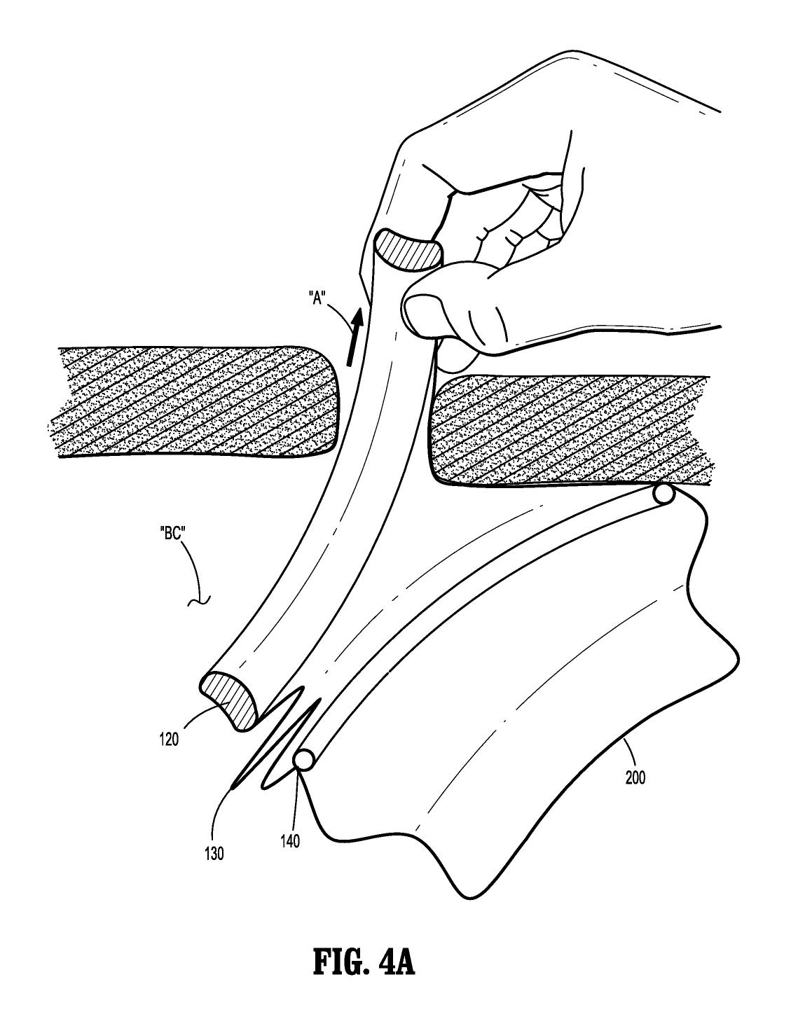

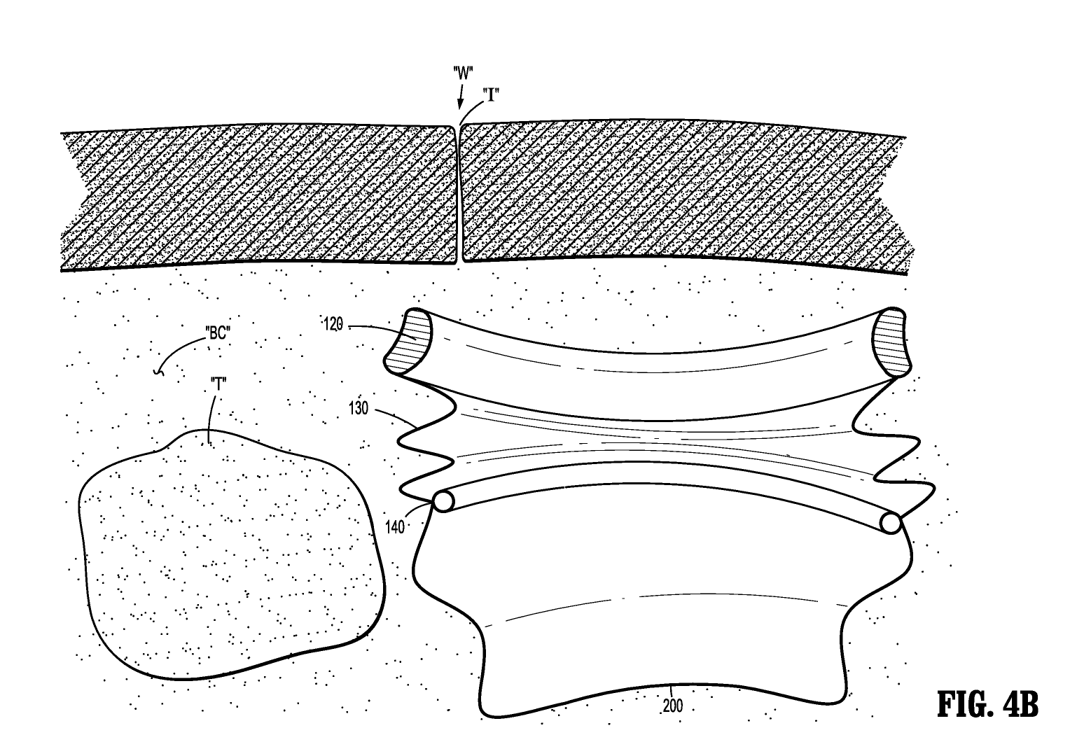

With reference to FIGS. 3-7 a method of operating the surgical apparatus 10 in accordance with the present disclosure will be described. As depicted in FIGS. 3-4B, in order to access a tissue specimen “T” positioned within a body cavity “BC” (FIG. 3), the surgeon first collapses and deforms the proximal ring 120 and the distal ring 140 of the wound retractor 100 into an oblong configuration for insertion into wound “W” through incision “I” (FIG. 4A). Once the wound retractor 100 and the specimen bag 200 are placed through incision “I” and the proximal ring 120 and the distal ring 140 are released, the proximal ring 120 and distal ring 140 resume their generally circular configuration so that the surgical apparatus 10 is adjacent the tissue specimen “T” to be removed from the body cavity (FIG. 4B). The surgeon can then introduce tissue specimen “T” through the proximal ring 120, the film 130, and the distal ring 140 into the specimen bag 200 by use of a forceps, grasper, or any other suitable medical device.

Once tissue specimen “T” has been introduced into the specimen bag 200, the surgeon then collapses and deforms the proximal ring 120 using a forceps, grasper, or similar instrument and withdraws the proximal ring 120 through the incision “I”. In other embodiments, the proximal ring 120 has a tether thereon (not shown), which may be pulled proximally by the surgeon to withdraw the proximal ring 120 from the body cavity “BC”. As depicted in FIG. 4A, the surgeon may pull the proximal ring 120 (indicated by arrows “A” in FIG. 4A) to remove the proximal ring 120 from the incision “I” after the specimen “T” is positioned within the specimen bag 200.

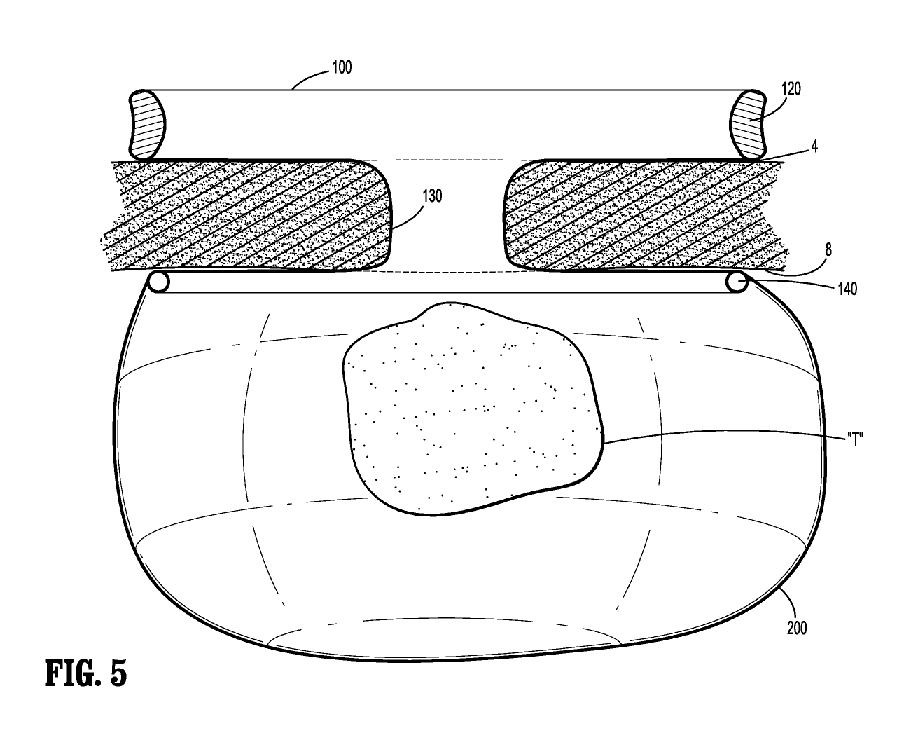

As shown in FIG. 5, the proximal ring 120 is removed from the body cavity through incision “I” so that it rests on the outer skin 4 adjacent incision “I” and the surgeon adjusts the film 130 and the proximal ring 120, e.g., pulling the proximal ring 120 proximally to tension the film 130, such that the distal ring 140 comes into abutment with an inner surface 8 of wound “W”. The distal ring 140 is positioned adjacent the inner surface 8 of wound “W”, and reverts to its generally circular configuration so that the generally circular opening of distal ring 140 encompasses the inner surface 8 of the wound “W” (FIG. 5).

With the distal ring 140 in abutment with the inner surface 8 of wound “W”, the proximal ring 120 is rolled distally towards the distal ring 140 and the outer surface 4 of wound “W” to a desired position, such that the film 130 is furled about the proximal ring 120 (not shown) and thereby tensioned.

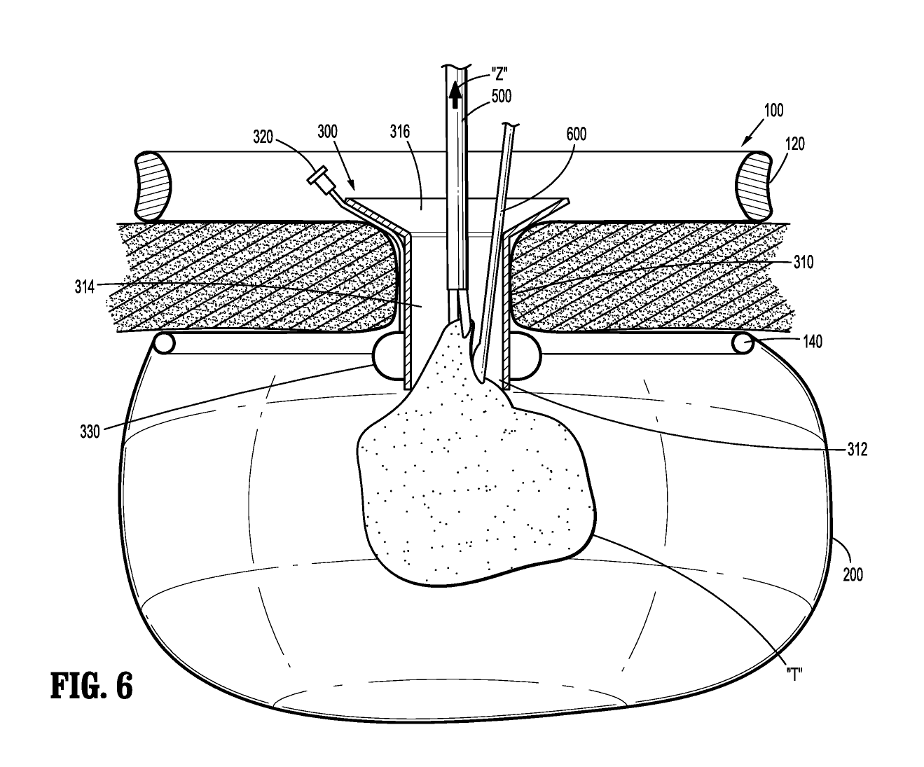

Referring to FIG. 6, once the wound retractor 100 and the specimen bag 200 are in position, surgical tools and instruments may pass through the wound retractor 100 with the distal ring 140 in abutment to the inner surface 8 of wound “W”, and the proximal ring 120 rolled to a desired position and/or brought into abutment to the outer surface 4 of wound “W”. The wound retractor 100 may be tensioned further to provide retraction of incision “I”, increasing the incision diameter.

Where the tissue specimen “T” in the specimen bag is too large to be removed through the incision, the wound guard 300 may be introduced through the wound retractor 100 (FIG. 6). Inflation gases are introduced through the inflation port 320 on the wound guard 300 to inflate the anchoring balloon 330 to secure the distal portion 312 of the wound guard 300 within the body cavity “BC”. In embodiments, inflation gases (not shown) are also introduced through the wound guard 300 to inflate the specimen bag 200.

In other embodiments, not shown, the wound guard 300 may be used with a surgical apparatus including the wound retractor described herein, but lacking the specimen bag attached thereto.

Referring briefly to FIG. 5, the wound guard 300 is introduced through the lumen 314 of wound retractor 100. FIG. 5 also depicts the anchoring balloon 330 in an inflated state, thereby anchoring the wound guard 300 at the site of the wound.

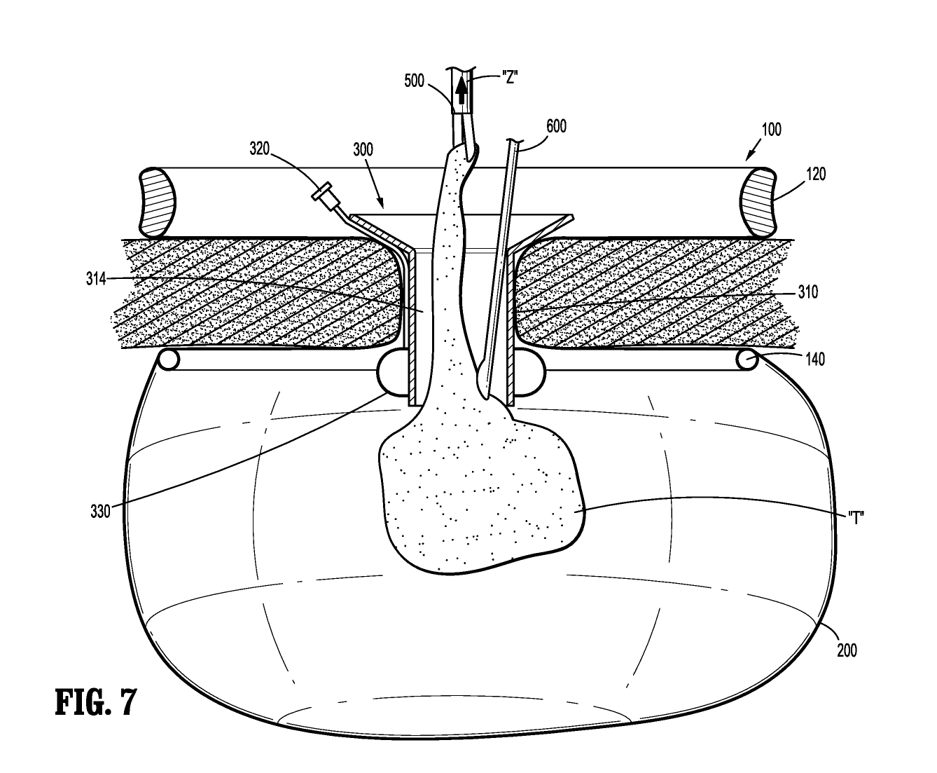

As depicted in FIGS. 6 and 7, a forceps 500, or any other grasper device, may then pass through the wound guard 300 into the specimen bag 200 and grasp the tissue specimen “T” therein.

The anchoring balloon 330 on the wound guard 300 helps stabilize the tissue specimen “T” during removal of the tissue specimen “T” from the specimen bag 200. As the forceps 500 are pulled proximally (indicated by arrows “Z” in FIGS. 6 and 7) to remove tissue specimen “T” from the specimen bag 200, the tissue specimen “T” passes proximally through the wound guard 300. To the extent the tissue specimen “T” is too large to pass through the lumen 314 of the wound guard 300, the rigid, hard materials utilized to form the wound guard 300 permit the use of morcellators, scalpels, knives, or similar devices to break up the tissue specimen “T” without damaging the wound retractor 100 and/or any body tissue encompassing the incision “I”. As illustrated, a scalpel 600 may be introduced through the wound guard 300 to break up the tissue specimen “T” as the tissue specimen “T is removed from the specimen bag 200.

The cut portions of the tissue specimen “T” may thus pass through the elongate shaft 310 of the wound guard 300 as it is removed from the specimen bag 200. In embodiments, if the tissue specimen “T” to be removed separates and portions thereof remain in the specimen bag 200 as tissue specimen “T” is cut, the surgeon may grab those additional pieces of tissue with a forceps or other grasper, and/or use a vacuum source for removal of any remaining portion of the tissue specimen “T” from the specimen bag 200. In other embodiments, the volume of the tissue specimen “T” in the specimen bag 200, as well as any fluids from the tissue specimen “T”, may be reduced to a point that specimen bag 200, with any remaining tissue and/or fluids therein, may pass through incision “I” without need for any further morcellation.

In other embodiments, not shown, power morcellators may be used with the wound retractor and specimen bag assembly 10 of the present disclosure.

Once a sufficient amount of the tissue specimen “T” is removed from the specimen bag 200 to facilitate removal of the specimen bag 200 through incision “I”, any inflation gases are withdrawn from the anchoring balloon 330. The surgeon then collapses and deforms the distal ring 140 using a forceps, grasper, or similar instrument and the distal ring 140 and specimen bag 200 are withdrawn through the incision “I”. In other embodiments, the distal ring 140 has a tether thereon (not shown), which may be pulled proximally by the surgeon to remove the distal ring 140 and the specimen bag 200 from the body cavity “BC”.

After the specimen bag 200 is removed, any tissue remaining therein can be removed from the specimen bag 200 for further examination or the specimen bag 200 can be discarded.

The specimen bags of the present disclosure provide safe tissue extraction at the end of minimally invasive surgical procedures. Diseased tissue may be removed from the body without seeding of spilled tissue cells inside the abdomen. The design of the wound retractor/specimen bags of the present disclosure, with the wound guard described above, allows for the surgeon to break up tissue without tearing the specimen bag and possibly releasing tissue contents back into the body of the patient. It is further envisioned that the methods of using the specimen bags of the present disclosure may be modified to accommodate needs of a given procedure and/or the preferences of the surgeon. It is further envisioned that the embodiments disclosed herein may be used to remove any tissue or object from the body.

It will be understood that various modifications may be made to the embodiments disclosed herein. For example, other methods for introducing specimen bags of the present disclosure into the body of a patient may be used. Additionally, other specimen bag shapes may be used. Further, the terminology of similar components with the various embodiments should not be construed as specific to any particular embodiment. Thus, the above description should not be construed as limiting, but merely as exemplifications of preferred embodiments. Those skilled in the art will envision other modifications within the scope and spirit of the claims appended hereto.

What is claimed is:

1. A kit comprising:
a wound retractor and specimen bag assembly including:
a proximal ring,
a distal ring,
a film disposed between the proximal ring and the distal ring, and
a specimen bag attached to the distal ring of the wound retractor; and
a wound guard.

2. The kit of claim 1, wherein the proximal ring of the wound retractor defines a generally circular opening and is deformable.

3. The kit of claim 1, wherein the distal ring of the wound retractor defines a generally circular opening and is deformable.

4. The kit of claim 1, wherein the film of the wound retractor defines an adjustable length between the proximal ring and the distal ring.

5. The kit of claim 1, wherein the specimen bag is attached to the distal ring of the wound retractor by a method selected from the group consisting of adhesive bonding, welding, heat-sealing, and combinations thereof.

6. The kit of claim 1, wherein the proximal ring, the distal ring, or both, possess a tether thereon.

7. The kit of claim 1, wherein the wound guard includes a distal portion defining a generally circular opening, a proximal portion defining a generally circular opening, and an elongate shaft defining a lumen between the distal portion and the proximal portion.

8. The kit of claim 1, wherein the wound guard possesses an anchoring balloon which is inflated to anchor the wound guard in a body opening.

9. The kit of claim 1, further including a grasping element for grasping tissue for removal from the specimen bag.

10. The kit of claim 1, further comprising a device for breaking up tissue as it is removed from the specimen bag.

11. The kit of claim 10, wherein the device for breaking up tissue is selected from the group consisting of a scalpel, a morcellator, a knife, and combinations thereof.

12. A method comprising:
inserting a wound retractor and specimen bag assembly having a proximal ring, a distal ring, a film disposed between the proximal ring and the distal ring, and a specimen bag attached to the distal ring, into a body cavity through a body opening;
passing a tissue specimen through an opening defined by the proximal ring, the film and the distal ring of the wound retractor into the specimen bag;
removing the proximal ring from the body cavity through the body opening so that the film is adjacent tissue encompassing the body opening;
pulling the proximal ring away from the distal ring such that the distal ring is brought into abutment with an inner surface of the tissue surrounding the body opening;
rolling the proximal ring about itself such that the film is furled about the proximal ring to bring the proximal ring into abutment with an outer surface of the tissue;
introducing a wound guard through the proximal ring into the body opening; and
removing the tissue specimen from the specimen bag.

13. The method of claim 12, further including rolling the proximal ring to retract the incision and bring the proximal ring into abutment with an outer surface of the tissue.

14. The method of claim 12, further including inflating an anchoring balloon on the wound guard to anchor the wound guard in the body opening.

15. The method of claim 12, further including:
introducing a grasping element through the wound guard into the specimen bag and grasping the tissue specimen therein;
proximally pulling the grasping element while breaking up the tissue specimen as the tissue specimen passes through the wound guard upon its removal from the specimen bag.

16. The method of claim 12, further including breaking up the tissue specimen prior to removing the tissue specimen from the specimen bag.

17. The method of claim 16, wherein breaking up the tissue specimen as it passes through the wound guard upon its removal from the specimen bag includes mechanically breaking up the tissue.
